(19) United States
(12) Patent Application Publication
(10) Pub. No.: US 2023/0181490 A1
(43) Pub. Date: Jun. 15, 2023

(54) PRO-LYCOPENE RICH COMPOSITION AND METHODS OF USING SAME

(71) Applicant: LYCORED LTD., Beer Sheva (IL)

(72) Inventors: Tal OFFER, Givat Ada (IL); Tanya SEDLOV, Beer Sheva (IL); Etgar LEVY-NISSENBAUM, Ramat HaSharon (IL); Oleg BRAVERMAN, Beer Sheva (IL)

(21) Appl. No.: 17/923,665

(22) PCT Filed: May 6, 2021
(86) PCT No.: PCT/IL2021/050524

§ 371 Date: Nov. 7, 2022

Related U.S. Application Data
(60) Provisional application No. 63/021,188, filed on May 7, 2020.

(51) Int. Cl. A61K 31/07; A61K 31/355; A61K 45/06

(52) U.S. Cl. CPC A61K 31/07; A61K 31/355; A61K 45/06

(57) ABSTRACT

The present invention is directed to a composition including 1-15% by weight pro-Lycopene, and methods of using same, such as for preventing or treating an oxidative stress related condition.

### CROSS-REFERENCE TO RELATED APPLICATIONS

This application claims the benefit of priority of U.S. Provisional Pat. Application No. 63/021,188, titled “PRO-LYCOPENE RICH COMPOSITION AND METHODS OF USING SAME”, filed May 7, 2020, the contents of which are incorporated herein by reference in their entirety.

### FIELD OF THE INVENTION

The invention relates generally to the field of carotenoids, and methods of using the same, such as for reducing oxidative stress.

### BACKGROUND

It is well established that protection against oxidative stress and UV radiation of a tissue, e.g., the skin, can be achieved by topical compositions of various protective ingredients. A particular group of protective compositions are intended for oral administration. Oral compositions contain active ingredients which are delivered to the tissue, e.g., skin, via an internal transport mechanism and thus protect the skin from oxidative stress and/or UV radiation damage. A particular group of active ingredients which are suitable for use with such oral compositions are carotenoids. The use of a mixture of carotenoids wherein canthaxanthin is the primary carotenoid in the composition have been described. However, the use of canthaxanthin is known to be limited due to adverse effects it may have on pigmentation. Also, different foodstuff and beverages intended for providing protection to the skin against UV sun radiation have been reported. These foodstuff and beverages comprise carotenoids as well as ascorbic acid, tocopherols, coenzyme Q10 and reduced glutathione. A composition for protecting skin against UV radiation and the harmful effects thereof, wherein the composition contains a pro-vitamin A carotenoid and lycopene, has been described. The use of such a composition is limited by the negative effect that pro-vitamin A carotenoids may have on the subject’s health at certain dosage levels. An excess of vitamin A, which is produced in the body from pro-vitamin A carotenoids, was found to have adverse effects on health. The protective effect of tomato paste, which is known to contain inter alia lycopene, beta-carotene and tocopherol, against UV light-induced erythema, was shown. However, a problem in achieving the desired carotenoid serum levels has been reported, and suggested it bears poor bioavailability.

Accordingly, there is a long felt need to develop a composition having antioxidant activity (e.g., capable of reducing oxidative stress), while having increased bioavailability.

### SUMMARY

According to a first aspect, there is provided a composition comprising pro-Lycopene in the amount of 1-15% by weight of said composition, and an acceptable carrier.

According to another aspect, there is provided a method for preventing or treating an oxidative stress related condition or disease in a subject in need thereof, comprising administering to the subject a therapeutically effective amount of the composition of the invention, thereby preventing or treating oxidative stress related condition or disease in the subj ect.

In some embodiments, the composition further comprises an additional carotenoid selected from the group consisting of: neurosporene, phytoene, phytofluene, zeta carotene, beta carotene, trans-Lycopene and any combination thereof.

In some embodiments, the pro-Lycopene constitutes at least 40% (w/w) of the total lycopene in the composition.

In some embodiments, the weight per weight ratio of the pro-Lycopene to the trans-Lycopene ranges from 1.5:1 (w/w) to 6:1 (w/w).

In some embodiments, the composition comprises neurosporene in the amount of 3-8% by weight of the composition.

In some embodiments, the composition comprises phytoene is in the amount of 40-50% by weight of the composition.

In some embodiments, the composition comprises phytofluene is in the amount of 10-20% by weight of the composition.

In some embodiments, the composition comprises zeta carotene is in the amount of 15-25% by weight of the composition.

In some embodiments, the weight per weight ratio of the phytoene to both the phytofluene and the zeta carotene ranges from 2:1 (w/w) to 4:1 (w/w).

In some embodiments, the composition comprises trans-Lycopene in the amount of less than 5% by weight of the composition, beta carotene in the amount of less than 3% by weight of the composition, or a combination thereof.

In some embodiments, the composition further comprises a tocopherol.

In some embodiments, the composition comprises tocopherol is in the amount of 2-5.5% by weight of the composition.

In some embodiments, the composition comprises pro-Lycopene in the amount of 10-20% (w/w) of the total carotenoids in the composition.

In some embodiments, the composition is for use in reducing oxidative stress.

In some embodiments, the administering comprises orally administering.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the figures and by study of the following detailed description.

### BRIEF DESCRIPTION OF THE FIGURES

FIG. 1 includes a graph showing the dose response of tangerine tomato extract (TG) and Control (e.g., Lyc-O-Mato) in antioxidant response element (ARE) induction in normal human dermal fibroblasts (NHDF).

FIGS. 2A-2B include vertical bar graphs showing the dose response of TG extract (2A) and Lyc-O-Mato (2B) in ARE induction in T47D breast cancer cells ; EA - Ethyl acetate extraction; SCE - supercritical extraction.

FIG. 3 includes a chromatogram of HPLC analysis of a TG supercritical extract. C30 chromatographic column was employed.

FIG. 4 includes a graph showing a variety of carotenoids spectra in tangerine, which may protect skin from UV/visible light irradiation in wide wavelength (WL) ranging from 240 to 520 nm. Phytoene (290 nm); Phytofluene (350 nm); z-carotene (400 nm); Neurosporene (440-470 nm); and Lycopene (450-520 nm).

### DETAILED DESCRIPTION

In some embodiments, the present invention is directed to a composition comprising high amount of prolycopene.

As used herein, the term “pro-Lycopene” refers to tetra-cis-lycopene, (7Z,9Z,7′Z,9′Z)-xi,xi-carotene, 7,9,7′,9′-tetracis-lycopene, or any combination thereof.

In some embodiments, supercritical extracts of Tangerine tomato can be standardized by total carotenoids amounts. In some embodiments, a standardized extract comprises total carotenoids amount of 10-30% by weight.

TABLE 1

A non-limiting example of a standardized extract

| Active ingredients | Specification |
|---|---|
| trans-Lycopene | 0.5-1.8% |
| tetra-cis-lycopene | 1-4% |
| Phytoene | 4-14% |
| Phytofluene | 1-4.5% |
| z-carotene | 2-7% |
| b-carotene | 0.1-0.3% |
| Tocopherols | 1-4% |
| gamma-carotene | 0.2-1% |
| Neurosporene | 2-8% |
| Total carotenoids | 10-30% |
| Phytoene & Phytofluene | 5-18.5% |
| Total lycopene | 1.5-5.8% |

In some embodiments, the composition of the invention comprises pro-Lycopene in the amount of: 3-10% by weight of the composition, 1-15% by weight of the composition, 3-8% by weight of the composition, 4-9% by weight of the composition, 1-6% by weight of the composition, 2-10% by weight of the composition, or 7-12% by weight of the composition. Each possibility represents a separate embodiment of the invention.

In some embodiments, the composition further comprises an additional carotenoid. As used herein, “an additional carotenoid” refers to any carotenoid or a metabolite thereof, other than or being different from pro-Lycopene. In some embodiments, the additional carotenoid is selected from: neurosporene phytoene, phytofluene, zeta carotene, beta carotene, trans-Lycopene, or any combination thereof.

In some embodiments, the additional carotenoid is neurosporene.

In some embodiments, pro-Lycopene constitutes at least 40% (w/w) of the total lycopene in the composition, at least 50% (w/w) of the total lycopene in the composition, at least 60% (w/w) of the total lycopene in the composition, at least 70% (w/w) of the total lycopene in the composition, at least 80% (w/w) of the total lycopene in the composition, or at least 90% (w/w) of the total lycopene in the composition, or any value and range therebetween. Each possibility represents a separate embodiment of the invention. In some embodiments, pro-Lycopene constitutes 40-90% (w/w) of the total lycopene in the composition, 40-80% (w/w) of the total lycopene in the composition, 50-75% (w/w) of the total lycopene in the composition, 35-75% (w/w) of the total lycopene in the composition, 60-95% (w/w) of the total lycopene in the composition, 55-80% (w/w) of the total lycopene in the composition, or 70-95% (w/w) of the total lycopene in the composition. Each possibility represents a separate embodiment of the invention.

As used herein, the phrase “total lycopene” refers to the amount, weight, quantity, concentration, or level of all pro- (e.g., cis-) and trans-Lycopene isomers combined.

In some embodiments, trans-Lycopene is all-trans-Lycopene.

In some embodiments, the weight per weight ratio of pro-Lycopene to trans-Lycopene ranges from 1.5:1 (w/w) to 6:1 (w/w), 1.5:1 (w/w) to 5:1 (w/w), 1.5:1 (w/w) to 4:1 (w/w), 1.5:1 (w/w) to 3:1 (w/w), or 1.5:1 (w/w) to 2:1 (w/w). Each possibility represents a separate embodiment of the invention.

In some embodiments, a carotenoid is a natural carotenoid extracted, isolated or purified from a fruit, a vegetable, or a plant (including any plant part). In another embodiment, a carotenoid is carotenoid extracted from a tomato plant. In another embodiment, a carotenoid is a carotenoid extracted from a tomato fruit. In another embodiment, a tomato carotenoid is a tomato extract enriched for a carotenoid. In another embodiment, tomato carotenoid is a carotenoid-rich tomato extract which is all-natural. In another embodiment, tomato carotenoid is a tomato carotenoid complex. In another embodiment, tomato carotenoid complex comprises a complex of phytonutrients including a plurality of carotenoids (such as phytoene, phytofluene, zeta carotene, beta-carotene, etc.), tocopherols and phytosterols. In some embodiments, a carotenoid is a synthetic carotenoid.

In some embodiments, the present invention provides a tomato extract obtained by an innovative extraction protocol. This particular extract which comprises pro-Lycopene (in amounts as specified hereinbelow), has increased bioavailability and anti-oxidative stress activity. In some embodiments, increased bioavailability and anti-oxidative stress activity is compared to other tomato extracts. In some embodiments, increased bioavailability and anti-oxidative stress activity enables to provide the composition of the invention to a subject in need, for example, at a lower dose without reducing the survival, wellbeing, or both, of the subject, while achieving superior anti-oxidative stress effect. In some embodiments, administering the composition of the invention to a subject in need enables to increase the efficacy of the treatment by providing the active ingredients, such as pro-Lycopene, while increasing the therapeutic effect (anti-oxidative stress effect), but without reducing the survival, wellbeing, or both, of the subject, resulting from the increased bioavailability of the pro-Lycopene.

In some embodiments, the composition of the invention provides greater amounts of carotenoids with greater bioavailability, e.g., pro-Lycopene, compared to other plant-, fruit-, or vegetable-derived extracts, such as a tomato. In some embodiments, the composition of the invention provides increased therapeutic efficacy with increased bioavailability compared to other plant-, fruit-, or vegetable-derived extracts, such as a tomato.

In some embodiments, the composition of the invention comprises natural carotenoids, synthetic carotenoids, or any combination thereof.

In some embodiments, the composition comprises phytoene in the amount of 40-60% by weight, 35-55% by weight, 40-55% by weight, 45-55% by weight, 40-50% by weight, or 30-60% by weight, of the composition. Each possibility represents a separate embodiment of the invention.

In some embodiments, the composition comprises phytofluene in the amount of 8-15% by weight, 10-20% by weight, 7-16% by weight, 12-19% by weight, 11-15% by weight, or 9-14% by weight of the composition. Each possibility represents a separate embodiment of the invention.

In some embodiments, the composition comprises neurosporene in the amount of 4-15% by weight, 10-20% by weight, 5-16% by weight, 11-19% by weight, 7-15% by weight, 2-10% by weight, or 3-14% by weight of the composition. Each possibility represents a separate embodiment of the invention.

In some embodiments, the composition comprises zeta carotene in the amount of 10-20% by weight, 12-24% by weight, 15-25% by weight, 16-28% by weight, 16-27% by weight, or 14-23% by weight of the composition. Each possibility represents a separate embodiment of the invention.

In some embodiments, the weight per weight ratio of phytoene to both phytofluene and to zeta carotene ranges from 2:1 (w/w) to 4:1 (w/w) or 2:1 (w/w) to 3:1 (w/w). Each possibility represents a separate embodiment of the invention.

In some embodiments, the composition comprises trans-Lycopene in the amount of less than 10% by weight, less than 7% by weight, less than 5% by weight, less than 3% by weight, less than 2% by weight, or less than 1% by weight, of the composition, or any value and range therebetween. Each possibility represents a separate embodiment of the invention. In some embodiments, the composition comprises trans-Lycopene in the amount of 1-3% by weight, 1-5% by weight, 2-6% by weight, 0.5-4.5% by weight, 0.1-3% by weight, 0.6-4.8% by weight, or 2.5-4% by weight of the composition. Each possibility represents a separate embodiment of the invention.

In some embodiments, the composition comprises beta carotene in the amount of less than 10% by weight, less than 7% by weight, less than 5% by weight, less than 3% by weight, less than 2% by weight, or less than 1% by weight, of the composition, or any value and range therebetween. Each possibility represents a separate embodiment of the invention. In some embodiments, the composition comprises beta carotene in the amount of 1-3% by weight, 1-5% by weight, 2-6% by weight, 0.5-4.5% by weight, 0.1-3% by weight, 0.6-4.8% by weight, or 2.5-4% by weight of the composition. Each possibility represents a separate embodiment of the invention.

In some embodiments, the composition comprises trans-Lycopene in the amount of less than 5% by weight of the composition, beta carotene in the amount of less than 3% by weight of the composition, or a combination thereof.

In some embodiments, the composition further comprises a tocopherol (e.g., vitamin E). In some embodiments, the composition comprises a tocopherol in the amount of 0.5-3% by weight, 1-5% by weight, 2-5.5% by weight, 4-6% by weight, 1.5-4.5% by weight, 3.5-5% by weight, or 2.5-4% by weight of the composition. Each possibility represents a separate embodiment of the invention.

Methods for determining the amounts of phytonutrients, such as carotenoids, are common and would be apparent to one of ordinary skill in the art. Non-limiting examples for such methods include, but are not limited to, gas chromatography, liquid chromatography, and mass spectrometry.

In some embodiments, there is provided a composition for use in reduction of oxidative stress.

As used herein, the term “oxidative stress” refers to an imbalance between the effect of reactive oxygen species (ROS) and the capability of a living system, e.g., a cell, a tissue, an organ, a subject, or combination thereof, to eliminate, e.g., detoxify, such ROS and/or to rectify the damage induced by same.

In some embodiments, the oxidative stress is induced by or a result of exposure to radiation. In some embodiments, the radiation comprises UV/visible light wavelength. In some embodiments, the UV radiation is UVA, UVB, UVC, or any combination thereof. In some embodiments, radiation comprises sunlight.

In some embodiments, radiation comprises any radiation wavelength within the light spectrum. As used herein, the term “light spectrum” encompasses wave lengths ranging from 10⁻⁹ m to 10⁻³ m. In some embodiments, radiation wavelength within the light spectrum comprises UV radiation, visible light radiation, infrared radiation, or a combination thereof. In some embodiments, an exposure to radiation comprises an exposure to sunlight.

As used herein, the term “ultraviolet (UV)” encompasses any wavelength of the UV range. In some embodiments, UV is UV radiation. In some embodiments, UV radiation is UVA radiation, UVB radiation, UVC, or any combination thereof.

In some embodiments, the composition is suitable for reducing oxidative stress induced by or resulting from exposure to sunlight. In some embodiments, the composition is suitable for reducing oxidative stress induced by or resulting from exposure to radiation at a continuous wavelength of 290 nm to 520 nm. In some embodiments, the composition is suitable for reducing oxidative stress induced by or resulting from exposure to radiation comprising a wavelength of 420 nm to 450 nm, 430 nm to 455 nm, 440 nm to 450 nm, 440 nm to 460 nm, 440 nm to 470 nm, 450 nm to 465 nm, or 445 nm to 475 nm.

Methods for determining oxidative stress levels are common and would be apparent to one of ordinary skill in the art. Non-limiting examples for such methods are reviewed by Katerji et al., 2019 (Oxidative Medicine and Cellular Longevity), as well as an ARE assay exemplified hereinbelow.

In some embodiments, the composition is an oral composition. In some embodiments, the composition is a pharmaceutical or a nutraceutical composition. In some embodiments, the composition is a topical composition. In some embodiments, the composition comprises a pharmaceutical or a nutraceutical acceptable carrier or excipient.

In some embodiments, an oral composition is in the form of a soft gel capsule, a tablet, a two-piece capsule, or an oral dispersible film (ODF). In some embodiments, an oral composition is in the form of a beverage, a shot, a gummy, or a powder. In some embodiments, an oral composition is mixed or assimilated into a food stuff, such as chocolate, ice cream, or others.

In some embodiments, a topical composition is in the form of an ointment, a cream, an oil, or a lotion.

In one embodiment, the composition of the invention can be provided to the individual per-se. In one embodiment, the composition of the present invention can be provided to the individual as part of a pharmaceutical composition or a nutraceutical composition comprising a pharmaceutical or a nutraceutical acceptable carrier.

In one embodiment, a “pharmaceutical composition” or a “nutraceutical composition” refers to a preparation of a composition as described herein with other chemical components such as physiologically suitable carriers and excipients. The purpose of a pharmaceutical composition or a nutraceutical composition is to facilitate administration of the composition to an organism.

In some embodiments, a process for producing a composition comprising pro-Lycopene in the amount of 3-10% by weight and an acceptable carrier, is provided. In some embodiments, the process comprises extracting a tangerine tomato as disclosed herein. In some embodiments, a composition of the invention comprises a tangerine tomato extract produced by the herein disclosed process.

In one embodiment, the phrases “physiologically acceptable carrier” and “pharmaceutically acceptable carrier” which be interchangeably used refer to a carrier or a diluent that does not cause significant irritation to a mammal and does not abrogate the biological activity and properties of the administered composition. An adjuvant is included under these phrases.

In one embodiment, “excipient” refers to an inert substance added to a composition to further facilitate administration of an active ingredient. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington’s Pharmaceutical Sciences,” Mack Publishing Co., Easton, PA, latest edition, which is incorporated herein by reference in its entirety.

In one embodiment, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

According to some embodiments, there is provided a method for treating or preventing an oxidative stress related condition or disease in a subject in need thereof, comprising administrating to the subject a therapeutically effective amount of the herein disclosed composition.

As used herein, “oxidative stress related condition or disease” encompasses any condition or disease which reduces a cell or a subject comprising same, wellbeing, survival, viability, functionality, or any combination thereof (e.g., “a pathological” condition), which involves oxidative stress as a part of its pathogenesis, pathophysiology, or both.

Non-limiting examples of oxidative stress related conditions or diseases include, but are not limited to, inflammation, cell proliferation related disease (e.g., cancer), neurodegenerative disease, and others.

In some embodiments, administering comprises orally administering.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject’s quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or formulations prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In some embodiments, preventing comprises reducing the disease severity, delaying the disease onset, reducing the disease cumulative incidence, or any combination thereof.

In some embodiments, the method comprises providing or selecting a subject in need of prevention of an oxidative stress related condition or disease.

According to some embodiments, there is provided a method for preventing an oxidative stress related condition or disease in a subject in need thereof, comprising the steps of: (a) providing a subject at risk of developing an oxidative stress related condition or disorder; and (b) administrating to the subject a therapeutically effective amount of a composition comprising pro-Lycopene in the amount of 1-15% by weight of the composition, and an acceptable carrier.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise”, or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6^{th} Edition) by Leroy “Skip” G Wade; Organic Chemistry by T. W. Graham Solomons and, Craig Fryhle.

### Materials and Methods

### Preparations of Extracts

Tangerine tomatoes (TG) were extracted under follow conditions: extraction type-supercritical CO₂ extraction (SCE), pressure 360 bar, and a temperature of 60° C. The amount of the crude extract from the raw material was 3.4% (w/w).

LycoMato extract was obtained by: (1) SCE, as described above, and (2) ethyl acetate extraction (EA), as previously described (WO2010082205A1).

A non-limiting example for the compounds identified within the supercritical extract (SCE), including their relative amounts, are specified hereinbelow (Table 2).

TABLE 2

Compounds identified within a TG extract obtained by a supercritical extraction

| Compound | Weight % |
|---|---|
| Trans-Lycopene | 2.37 |
| Pro-lycopene | 4.00 |
| Phytoene | 22.80 |
| Phytofluene | 7.67 |
| zeta-carotene | 13.00 |
| beta-carotene | 0.53 |
| Tocopherols | 2.69 |
| Total carotenoids | 50.37 |

### Content in TG Compared to LycoMato

A non-limiting example of a comparison of the active ingredients of TG (extract of the present invention) and Lyc-O-Mato 6% (both SCE and EA) is presented in Table 3.

TABLE 3

Comparison of phytonutrients content

| Active ingredients | Concentration, % | | |
|---|---|---|---|
| | Supercritical extraction | | EA extraction |
| | TG (extract of the present invention) | LycoMato | LycoMato |
| Pro-Lycopene | 4.00 | 0.00 | 0.00 |
| trans-Lycopene | 2.37 | 5.51 | 6.49 |
| Phytoene | 22.80 | 1.95 | 2.58 |
| Phytofluene | 7.67 | 0.46 | 0.60 |
| zeta-carotene | 13.00 | 0.11 | 0.12 |
| Neurosporene | 4.00 | 0.00 | 0.00 |
| beta-carotene | 0.53 | 0.48 | 0.18 |
| Tocopherols | 2.69 | 2.16 | 1.77 |
| Total Carotenoids | 50.37 | 8.51 | 9.98 |

### Cells

Normal human dermal fibroblasts (NHDF) were purchased from PromoCell GmbH (Heidelberg, Germany). The cells were grown in fibroblast growth medium 2 (PromoCell) according to the manufacturer’s instructions.

The T47D human mammary cancer cells grown in DMEM containing penicillin (100 U/ml), streptomycin (0.1 mg/ml), nystatin (12.5 µg/ml), 10% FCS and human recombinant insulin (6 µg/ml) in a humidified atmosphere with 5% CO₂.

### Transient Transfection and ARE Reporter Gene Assay

Cells were transfected using jetPEI reagent (Polyplus Transfection, Illkrich, France) in 24-well plates. NHDF primary human dermal fibroblasts or T47D human breast cancer cells (80,000 cells per well) were transfected with 0.2 µg 4×ARE reporter construct and 0.1 µg normalizing plasmid. Cells were seeded in culture media containing 3% fetal calf serum (FCS). The next day, cells were rinsed once with the appropriate culture medium, followed by addition of 0.45 ml of medium and 50 µl of DNA mixed with jetPEI. Cells were then incubated for 4-6 h at 37° C. The used medium was replaced with a fresh medium supplemented with 3% FCS including the tested compounds, and cells were incubated for additional 16-20 hr. ARE/Nrf2 reporter activity was measured in cell extracts and normalized to Renilla luciferase using a dual luciferase reporter assay system (Promega, Madison, WI, USA) according to the manufacturer’s instructions. The reporter construct used was the 4xARE reporter construct. Renilla luciferase (P-RL-null) expression vector served as internal transfection standards (Promega, Madison, WI, USA).

### Example 1

### Tangerine Tomato Extract Increases Anti Oxidative Stress Response in Cells

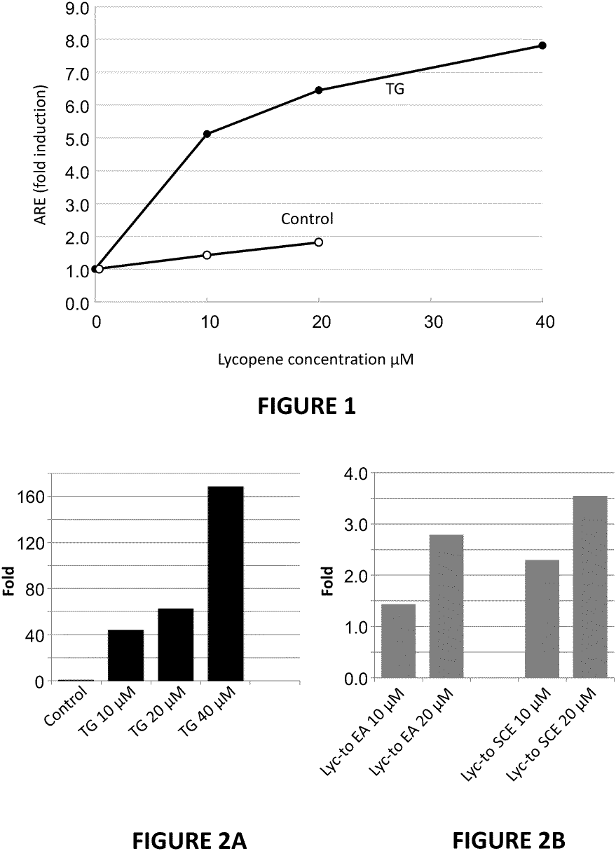

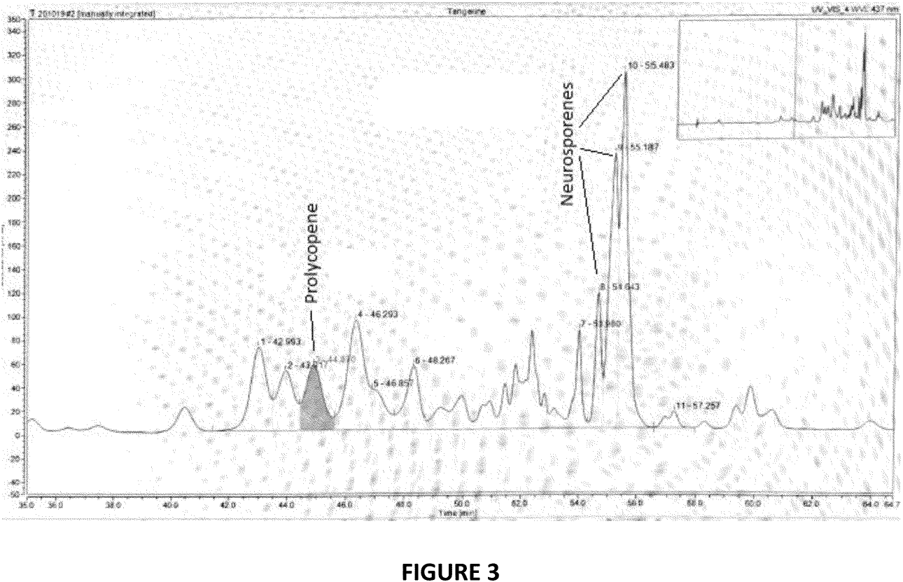

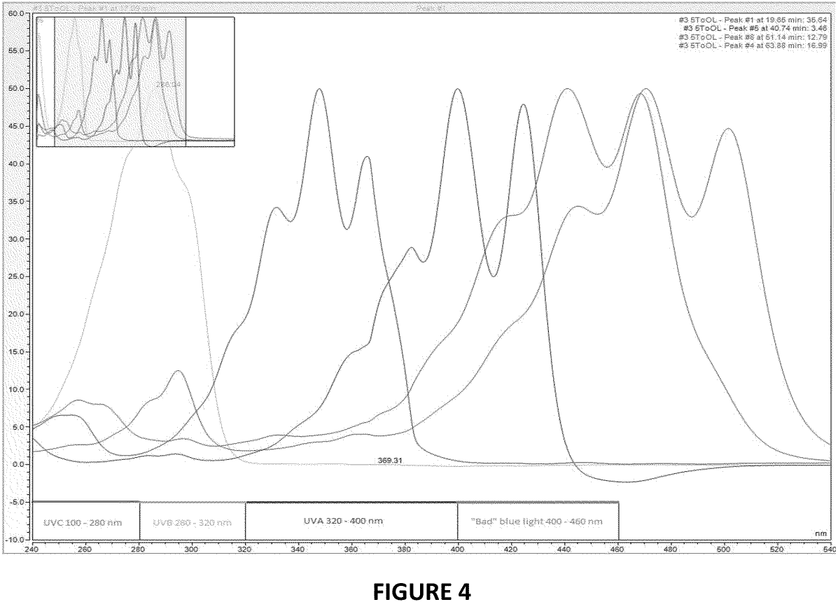

The fold induction of ARE by TG extract in NHDF dermal fibroblasts was found to be about 3-fold higher than by Lyc-O-Mato when compared on the basis of equal concentrations of lycopene (FIG. 1). Antioxidant response element (ARE) activity was rather low in this type of cells, and thus the inventors compared the activity of the two extracts in T47D breast cancer cells which show higher activity. The induction of ARE in this type of cells was found to be dose dependent, wherein the induction by TG extract was about 60-fold greater at 20 µM lycopene and about 160-fold greater at 40 µM lycopene, compared to Lyc-O-Mato control.

The induction of ARE, which is a marker of the antioxidant defense system of the cell, was about 20 times higher with TG extract than with Lyc-O-Mato control. Even if the comparison would have been performed based on the total amount of carotenoids, which is 6 times higher in the TG extract, there would still be about 3-fold higher activity in the TG extract, suggesting that the antioxidant protection of cells by the TG extract in this experimental set-up, is superior to that of Lyc-O-Mato.

### Example 2

### TG Extract Has Increased Bioavailability

A pharmacokinetics experiment is performed. Cross-over supplementation with TG or LycoMato capsules is performed. Two groups are compared: (1) TG tomato capsules comprising: 15 mg Lycopene (~1:2 (w/w) trans-lycopene to tetra-cis lycopene (pro); 41.5 mg Phytoene; 13.3 mg Phytofluene; 20.4 mg zeta-carotene; 0.6 mg β-carotene; and 5.5 mg tocopherols; and (2) Lyco-Mato capsules comprising: 15 mg trans-Lycopene; 6.5 mg Phytoene; 1.5 mg Phytofluene; 0.3 mg zeta-carotene; 0.45 mg β-carotene; and 5.1 mg tocopherols.

Experimental designs:

Experiment I

Steady state supplement experiment (4 blood samples):

| Step | Blood withdrawal | Day |
|---|---|---|
| 1 | Time zero of washout | Day -14 |
| 2 | Time zero of experiment | Day 1 |
| 3 | 3-day treatment (TG/LycoMato) | Day 4 |
| 4 | 6-day treatment (TG/LycoMato) | Day 7 |
| 5 | 9-day treatment (TG/LycoMato) | Day 10 |
| 6 | 13-day treatment (TG/LycoMato) | Day 14 |
| | Washout | |
| 7 | Time zero of cross experiment | Day 1 |
| 8 | 3-day treatment (LycoMato/TG) | Day 4 |
| 9 | 6-day treatment (LycoMato/TG) | Day 7 |
| 10 | 9-day treatment (LycoMato/TG) | Day 10 |
| 11 | 13-day treatment (LycoMato/TG) | Day 14 |

Experiment II

Steady state supplement experiment (3 blood samples)

| Step | Blood withdrawal | Day |
|---|---|---|
| 1 | Time zero of washout | Day -14 |
| 2 | Time zero of experiment | Day 1 |
| 3 | 3-day treatment (TG/LycoMato) | Day 4 |
| 4 | 6-day treatment (TG/LycoMato) | Day 7 |
| 5 | 9-day treatment (TG/LycoMato) | Day 10 |
| | Washout | |
| 6 | Time zero of cross experiment | Day 1 |
| 7 | 3-day treatment (LycoMato/TG) | Day 4 |
| 8 | 7-day treatment (LycoMato/TG) | Day 8 |
| 9 | 10-day treatment (LycoMato/TG) | Day 11 |

Plasma levels of: Lycopene, Phytoene, Phytofluene, zeta-carotene, and β-carotene, are determined.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A composition comprising pro-Lycopene in the amount of 1-15% by weight of said composition, and an acceptable carrier.

2. The composition of claim 1, further comprising an additional carotenoid selected from the group consisting of: neurosporene, phytoene, phytofluene, zeta carotene, beta carotene, trans-Lycopene and any combination thereof.

3. The composition of claim 1, wherein said pro-Lycopene constitutes at least 40% (w/w) of the total lycopene in said composition.

4. The composition of claim 1, wherein the weight per weight ratio of said pro-Lycopene to said trans-Lycopene ranges from 1.5:1 (w/w) to 6:1 (w/w).

5. The composition of claim 2, comprising said neurosporene in the amount of 3-8% by weight of said composition.

6. The composition of claim 2, comprising said phytoene is in the amount of 40-50% by weight of said composition.

7. The composition of claim 2,comprising said phytofluene is in the amount of 10-20% by weight of said composition.

8. The composition of claim 2, comprising said zeta carotene is in the amount of 15-25% by weight of said composition.

9. The composition of claim 2, wherein the weight per weight ratio of said phytoene to both said phytofluene and said zeta carotene ranges from 2:1 (w/w) to 4:1 (w/w).

10. The composition of claim 2,comprising said trans-Lycopene in the amount of less than 5% by weight of said composition, said beta carotene in the amount of less than 3% by weight of said composition, or a combination thereof.

11. The composition of claim 1, further comprising a tocopherol.

12. The composition of claim 11, wherein said tocopherol is in the amount of 2-5.5% by weight of said composition.

13. The composition of claim 1, comprising said pro-Lycopene in the amount of 10-20% (w/w) of the total carotenoids in said composition.

14. (canceled)

15. A method for preventing or treating an oxidative stress related condition or disease in a subject in need thereof, comprising administering to said subject a therapeutically effective amount of the composition of claim 1, thereby preventing or treating oxidative stress related condition or disease in the subject.

16. The method of claim 15, wherein said administering comprises orally administering.

17. The method of claim 15 or 16, wherein said oxidative stress is induced by radiation.

18. The method of claim 17, wherein said radiation comprises UV radiation, visible light radiation, infrared radiation, or any combination thereof.

19. The method of claim 18, wherein said UV radiation is UVA, UVB, UVC, or any combination thereof.
